(19) United States
(12) Patent Application Publication
(10) Pub. No.: US 2011/0288588 A1
(43) Pub. Date: Nov. 24, 2011

(54) SYSTEM AND METHOD FOR FACET FIXATION AND FUSION

(75) Inventors: Kingsley R. Chin, Wilton Manors, FL (US); Matthew Ibarra, LAKEWOOD, CA (US); Craig Henshaw, CHARLESTOWN, MA (US); Vito Lore, SOMERVILLE, MA (US); Kevin Chappuis, Randolph, MA (US); John Sullivan, Pelham, NH (US)

(73) Assignee: SPINEFRONTIER INC, Beverly, MA (US)

(21) Appl. No.: 13/111,574

(22) Filed: May 19, 2011

Related U.S. Application Data
(60) Provisional application No. 61/346,523, filed on May 20, 2010.

(51) Int. Cl. A61B 17/70

(52) U.S. Cl. 606/247; 606/264

(57) ABSTRACT

A spinal fixation and fusion assembly includes a bone anchoring member, a cylindrical rod and a cap. The bone anchoring member comprises an elongated body that is made entirely of bone type material. The elongated body includes a main shaft, a conical shaped distal end, a flared out proximal end, and a through-opening extending along an axis from the proximal end to the distal end. The cylindrical rod is shaped and dimensioned to be received within the through-opening and is made entirely of metal. The cap is made entirely of metal and is attached to a proximal end of the cylindrical rod.

### CROSS REFERENCE TO RELATED CO-PENDING APPLICATIONS

This application claims the benefit of U.S. provisional application Ser. No. 61/346,523 filed May 20, 2010 and entitled “SYSTEM AND METHOD FOR FACET FUSION”, the contents of which are expressly incorporated herein by reference.

### FIELD OF THE INVENTION

The present invention relates to a system and a method for facet fixation and fusion, and more particularly to a system and a method that utilizes a bone allograft product for bone-to-bone facet fusion.

### BACKGROUND OF THE INVENTION

The human spine includes individual vertebras that are connected to each other. Under normal circumstances the structures that make up the spine function to protect the neural structures and to allow us to stand erect, bear axial loads, and be flexible for bending and rotation. However, disorders of the spine occur when one or more of these spine structures are abnormal. In these pathologic circumstances, surgery may be tried to restore the spine to normal, achieve stability, protect the neural structures, or to relief the patient of discomfort. The goal of spine surgery for a multitude of spinal disorders especially those causing compression of the neural structures is often decompression of the neural elements and/or fusion of adjacent vertebral segments. Fusion works well because it stops pain due to movement at the facet joints or intervertebral discs, holds the spine in place after correcting deformity, and prevents instability and or deformity of the spine after spine procedures such as discectomies, laminectomies or corpectomies. Discectomy and fusion or corpectomy and fusion are most commonly performed in the cervical spine but there is increasing application in the thoracic and lumbar spine, as well.

Several spinal fixation systems exist for stabilizing the spine so that bony fusion is achieved. The majority of these fixation systems utilize fixation elements such as rods wires or plates that attach to screws threaded into the vertebral bodies, facets or the pedicles. In some fixation systems the facet joints are compressed together and attached together via spinal fixation elements 82a, 82b, shown in FIG. 1. However, in most prior art methods of facet fixation, compression and fixation does not result in actual bone-to-bone contact and fusion between superior and inferior facets. Accordingly, there is a need for a system and a method for bone-to-bone facet fixation that results in facet fusion between superior and inferior facets and spinal stabilization.

### SUMMARY OF THE INVENTION

The present invention relates to facet fixation and fusion methods and facet fixation and fusion devices, and in particular to cylindrical or multi-faceted components made of bone allograft material for the achievement of bone-to-bone facet fixation.

In general, in one aspect, the invention features a spinal fixation and fusion assembly including a bone anchoring member, a cylindrical rod and a cap. The bone anchoring member comprises an elongated body that is made entirely of bone type material. The elongated body includes a main shaft, a conical shaped distal end, a flared out proximal end, and a through opening extending along an axis from the proximal end to the distal end. The cylindrical rod is shaped and dimensioned to be received within the through opening and is made entirely of metal. The cap is made entirely of metal and is attached to a proximal end of the cylindrical rod.

Implementations of this aspect of the invention may include one or more of the following features. The elongated body may have a cylindrical cross section, rectangular cross section, or polygonal cross section. The elongated body may include screw threads, spikes, teeth, barbs, bumps, indentations, straight protrusions, helical protrusions, or combinations thereof. The main shaft may have a parallelepiped shape, the distal end may be an inverted truncated rectangular pyramid extending from the bottom surface of the main shaft, and the proximal end may be an inverted truncated rectangular pyramid extending from the top surface of the main shaft. The main shaft may have a polygonal shape, the distal end may be an inverted truncated polygonal pyramid extending from the bottom surface of the main shaft, and the proximal end may be an inverted truncated polygonal pyramid extending from the top surface of the main shaft. The spinal fixation assembly may further include a conical cap made entirely of metal and being attached to a distal end of the cylindrical rod. The conical cap comprises screw threads. The bone type material may be one of allograft bone material, biocompatible materials, synthetic bone growth promoting material, bone-polymer composite material, autograft bone material, xenograft bone material, polymers, resorbable material, non-resorbable material, or combinations thereof. The metal may be one of titanium, cobalt, stainless steel, chrome, alloys thereof, shape-memory alloy, ceramic-metallic composite materials, or combinations thereof.

In general, in another aspect, the invention features a spinal fixation and fusion assembly including a bone anchoring member and a cylindrical member. The bone anchoring member includes an elongated body made entirely of metallic material. The elongated body comprises a threaded portion at the distal end, a head at the proximal end, and a lag portion extending between the threaded portion and the head. The cylindrical member surrounds the lag portion of the bone—anchoring member and is made entirely of bone type material. The cylindrical member comprises a through opening extending along an axis from its proximal end to the distal end and the through opening includes inner threads and is dimensioned to receive said lag portion of the bone anchoring member. The spinal fixation may further include a polyaxial washer surrounding the head of the bone anchoring member, and the polyaxial washer includes spikes extending from its bottom surface.

In general, in another aspect, the invention features a spinal fixation and fusion assembly including a bone anchoring member and a cylindrical member. The bone anchoring member includes an elongated body and the elongated body is made entirely of bone type material. The elongated body comprises a threaded portion at the distal end, a head at the proximal end, and a lag portion extending between the threaded portion and the head. The cylindrical member surrounds the lag portion of the bone—anchoring member and is made entirely of bone type material. The cylindrical member comprises a through opening extending along an axis from its proximal end to the distal end and the through opening comprises inner threads and is dimensioned to receive said lag portion of the bone anchoring member.

In general, in another aspect, the invention features a spinal fixation and fusion method including forming an opening extending through first and second adjacent vertebral bodies and then inserting a spinal fixation and fusion assembly into the formed opening. The spinal fixation and fusion assembly comprises a bone anchoring member, a cylindrical rod and a cap. The bone anchoring member comprises an elongated body comprised entirely of bone type material and the elongated body comprises a main shaft, a conical shaped distal end, a flared out proximal end, and a through opening extending along an axis from the proximal end to the distal end. The cylindrical rod is shaped and dimensioned to be received within the through opening and is comprised entirely of metal and the cap is comprised entirely of metal and is attached to a proximal end of the cylindrical rod.

In general, in another aspect, the invention features a spinal fixation and fusion method comprising forming an opening extending through first and second adjacent vertebral bodies and inserting a spinal fixation and fusion assembly into said formed opening. The spinal fixation and fusion assembly comprises a bone anchoring member and a cylindrical member. The bone anchoring member comprises an elongated body comprised entirely of metallic material. The elongated body comprises a threaded portion at the distal end, a head at the proximal end, and a lag portion extending between the threaded portion and the head. The cylindrical member surrounds the lag portion of the bone anchoring member. The cylindrical member is comprised entirely of bone type material and comprises a through opening extending along an axis from its proximal end to the distal end and the through opening comprises inner threads and is dimensioned to receive said lag portion of the bone anchoring member. The spinal fixation and fusion assembly further includes a polyaxial washer surrounding the head of the bone anchoring member, and the polyaxial washer comprises spikes extending from its bottom surface.

In general, in another aspect, the invention features a spinal fixation and fusion method comprising forming an opening extending through first and second adjacent vertebral bodies and then inserting a spinal fixation and fusion assembly into said formed opening. The spinal fixation and fusion assembly comprises a bone anchoring member and a cylindrical member. The bone anchoring member comprises an elongated body comprised entirely of bone type material. The elongated body comprises a threaded portion at the distal end, a head at the proximal end, and a lag portion extending between the threaded portion and the head. The cylindrical member surrounds the lag portion of the bone anchoring member. The cylindrical member is comprised entirely of bone type material and comprises a through opening extending along an axis from its proximal end to the distal end and the through opening comprises inner threads and is dimensioned to receive said lag portion of the bone anchoring member.

In general, in another aspect, the invention features a facet fixation and fusion method comprising forming a first opening extending through a facet joint of first and second adjacent vertebral bodies and then forming a second opening extending through the first and second adjacent vertebral bodies. The second opening is adjacent to the first opening. Next, inserting a first spinal fixation assembly into the first opening, and then inserting a second spinal fixation and fusion assembly into the second opening. The first fixation assembly comprises a bone anchoring member and a polyaxial washer. The bone anchoring member comprises an elongated body having a threaded portion at the distal end, a head at the proximal end, and a lag portion extending between the threaded portion and the head. The polyaxial washer surrounds the head. The first fixation assembly is comprised entirely of metallic material. The second fixation and fusion assembly comprises a cylindrical elongated body comprised entirely of bone type material. The first and second openings may intersect each other, or may be non-intersecting. The first and second openings may have parallel or non-parallel trajectories. The first and second openings are formed through a double-barreled cannula.

The details of one or more embodiments of the invention are set forth in the accompanying drawings and description below. Other features, objects and advantages of the invention will be apparent from the following description of the preferred embodiments, the drawings and from the claims.

### BRIEF DESCRIPTION OF THE DRAWINGS

Referring to the figures, wherein like numerals represent like parts throughout the several views:

FIG. 1 depicts a prior art system for face fixation;

FIG. 1a and FIG. 1b are perspective views of a first embodiment of the spine fixation and fusion device according to this invention;

FIG. 2a and FIG. 2b are perspective views of the second embodiment of the spine fixation and fusion device according to this invention;

FIG. 3a, FIG. 3b, and FIG. 3c are perspective views of a third embodiment of the spine fixation device according to this invention;

FIG. 4a and FIG. 4b are perspective views of the fourth embodiment of the spine fixation and fusion device according to this invention;

FIG. 5a and FIG. 5b are perspective views of the fifth embodiment of the spine fixation and fusion device according to this invention;

FIG. 6a is a perspective view of the sixth embodiment of the spine fixation and fusion device according to this invention;

FIG. 6b is a perspective view of the embodiment of FIG. 6a having a metal cap at the top end;

FIG. 6c is a side cross-sectional view of the embodiment of FIG. 6b;

FIG. 7a and FIG. 7b are perspective views of the seventh embodiment of the spine fixation and fusion device according to this invention;

FIG. 8 is a perspective view of the eighth embodiment of the spine fixation and fusion device according to this invention;

FIG. 9a and FIG. 9b are perspective views of the ninth embodiment of the spine fixation and fusion device according to this invention;

FIG. 9c is a cross-sectional side view showing the installation of the spine fixation device of FIG. 9b;

FIG. 9d is a cross-sectional side view showing the fully installed spine fixation device of FIG. 9b;

FIG. 10a and FIG. 10b are an exploded view and perspective view of a tenth embodiment the spine fixation and fusion device according to this invention, respectively;

FIG. 11a through FIG. 11e are perspective views of the first method steps of inserting the fixation and fusion device of this invention in and through the facet joint;

FIG. 12a through FIG. 12h are perspective views of the second method of inserting the fixation and fusion device of this invention in and through the facet joint;

FIG. 13a through FIG. 13i are perspective views of the third method of inserting the fixation and fusion device of this invention in and through the facet joint; and

FIG. 14 is a perspective view of inserting a fixation and fusion device through the facet joint in a more lateral trajectory.

### DETAILED DESCRIPTION OF THE INVENTION

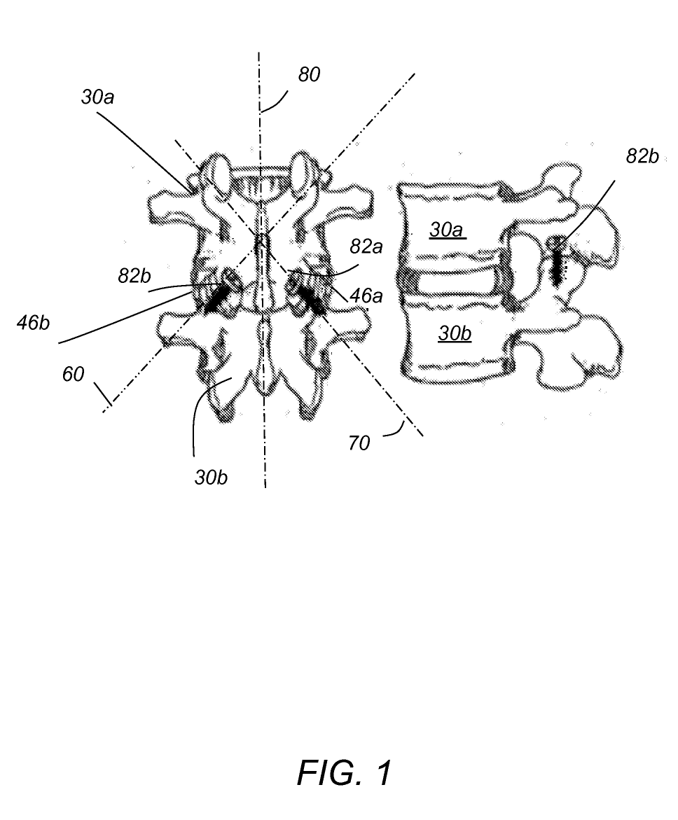

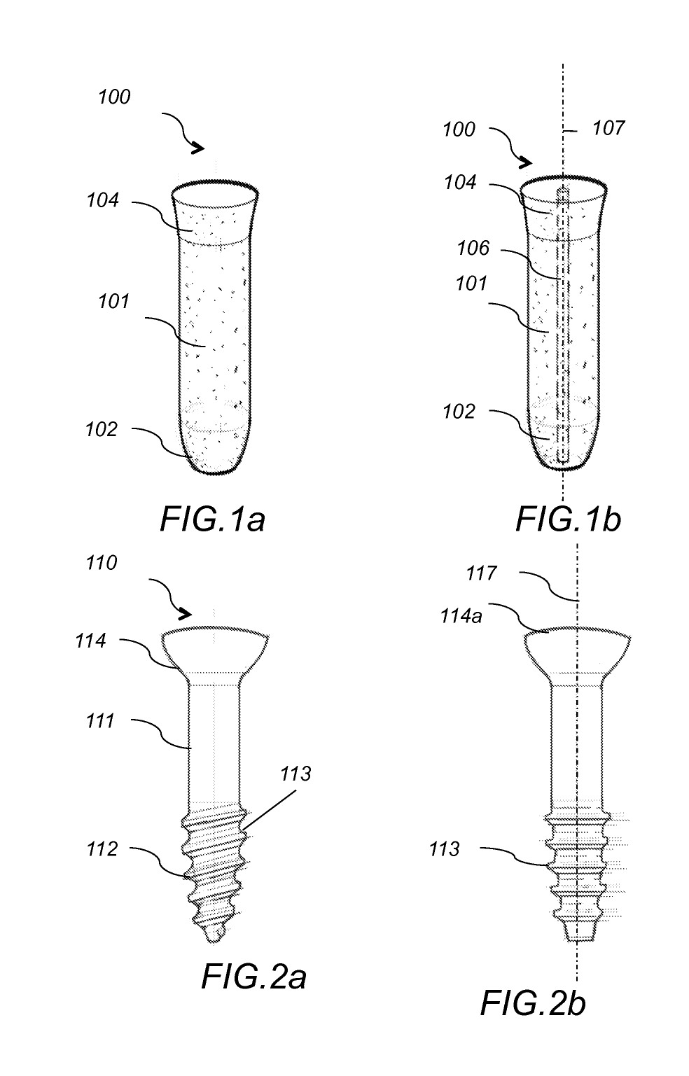

Referring to FIG. 1, spinal fixation elements 82a, 82b are used to secure together first and second facet joints 46a, 46b of two adjacent vertebras 30a and 30b. The spinal fixation elements 82a, 82b are inserted along directions 60, 70, respectively. In most cases, directions 60, 70 are symmetrically positioned to the left and right of the spinal midline 80. In this prior art example, fixation elements 82a, 82b are facet screws and are placed in a trans-facet way for connecting adjacent vertebras 30a, 30b. In other examples, fixation elements 82a, 82b, may be staples, wires, or pins, and they may connect adjacent or non-adjacent vertebras via trans-facet, intra-facet, trans-laminar, trans-facet-pedicular, trans-pedicular, or through any other vertebral location.

The present invention describes a new facet fixation and fusion device that is shaped and formed to be implanted into the vertebrae through the facet joint and into the pedicle in order to provide both spine fixation and fusion through the facet joint. The new facet fixation and fusion device is made of allograft material, which is actual bone material harvested from human donors.

Referring to FIG. 1a, a new facet fixation and fusion device comprises a cylindrical dowel 100 made of allograft bone material. The cylindrical dowel 100 includes an elongated cylindrical body 101 having a rounded conical shaped distal end portion 102 and a flared out proximal end portion 104. Distal end portion 102 tapers to a smaller diameter than the diameter of the cylindrical body 101. In the embodiment of FIG. 1b, cylindrical dowel 100 includes a through opening extending along axis 107 from the proximal end 104 to the distal end 102.

Referring to FIG. 2a, a new facet fixation and fusion device comprises a cylindrical dowel 110 made of allograft bone material. The cylindrical dowel 110 includes an elongated cylindrical body 111 having a conical shaped distal end portion 112 and a flared out conical head portion 114. Distal end portion 112 tapers to a smaller diameter than the diameter of the cylindrical body 111 and includes threads 113 that are designed to screw the dowel 110 into an opening extending through the facet joints, as will be described below. Head portion 114 has a flat top 114a and a diameter at the top larger than the diameter of the cylindrical body 111. In the embodiment of FIG. 2b, cylindrical dowel 110 includes threads 113 that are oriented perpendicular to the dowels main axis 117.

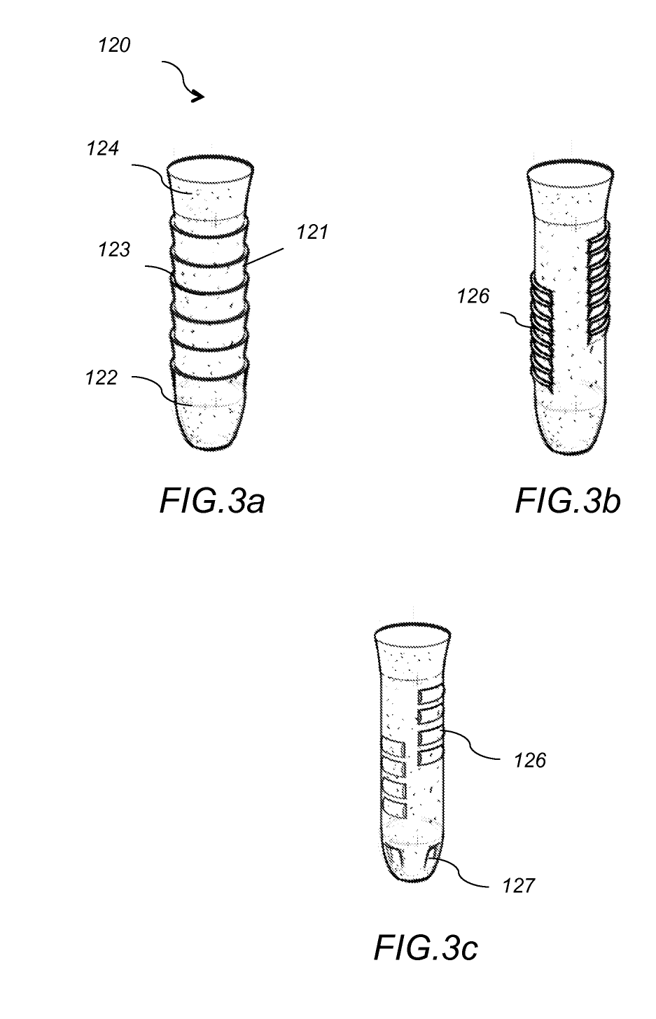

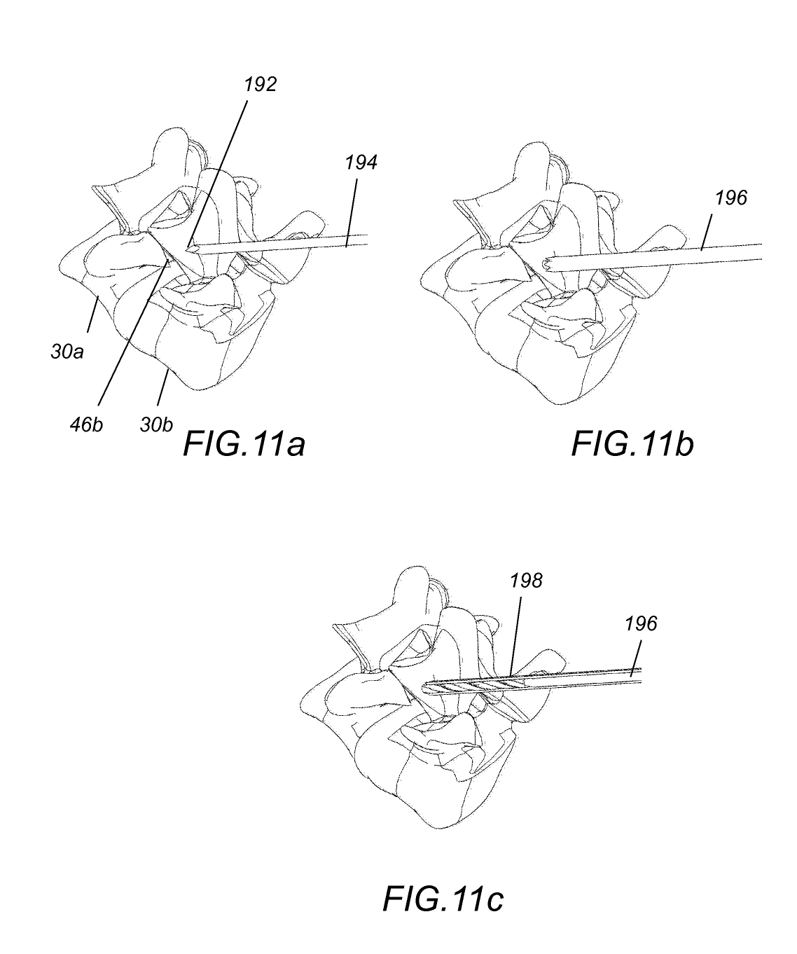

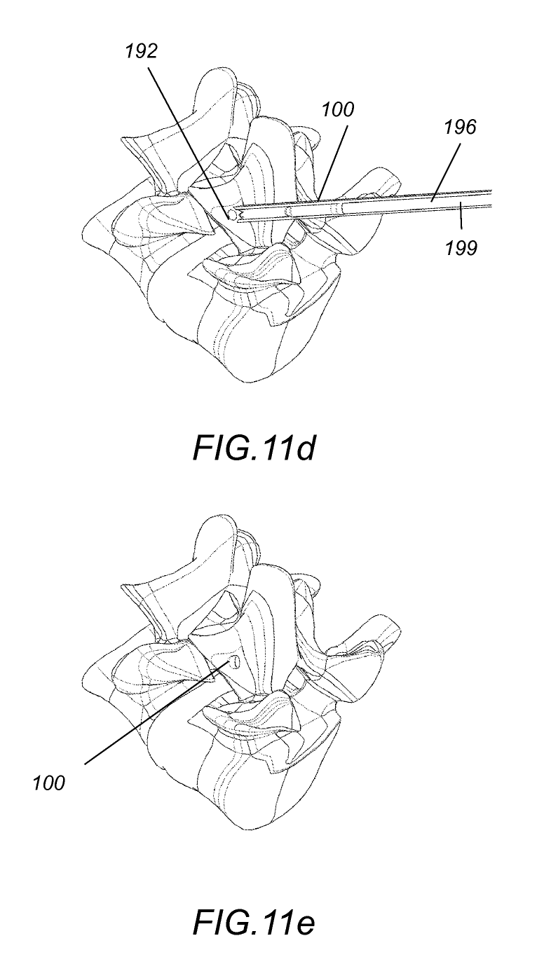

Referring to FIG. 3a, a new facet fixation and fusion device comprises a cylindrical dowel 120 made of allograft bone material. The cylindrical dowel 120 includes an elongated cylindrical body 121 having a rounded conical shaped distal end portion 122 and a flared out proximal end portion 124. Cylindrical body 121 includes barbs 123 or bumps 126 extending throughout the entire body 121 or covering only segments of the cylindrical body, as shown in FIGS. 3b and 3c. Distal end portion 122 tapers to a smaller diameter than the diameter of the cylindrical body 121. In the embodiment of FIG. 3c, distal end portion 122 includes indentations 127. In the embodiments of FIG. 5a and FIG. 5b, cylindrical body 121 includes elongated straight or helical patterned protrusions 129, 128 surrounding the elongated body, respectively. In one example, protrusions 128, 129 are crush ribs that dig into the side walls of the opening 192 made in the facet joint, shown in FIG. 11d.

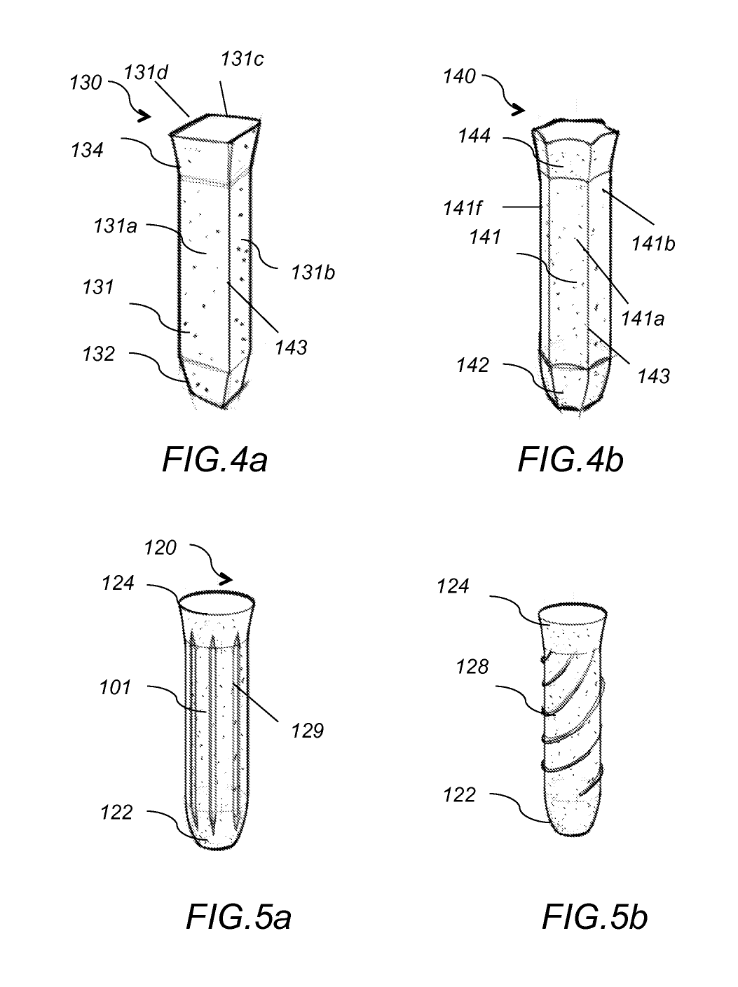

Referring to FIG. 4a, a new facet fixation and fusion device comprises a parallelepiped-shaped dowel 130 made of allograft bone material. The parallelepiped-shaped dowel 130 includes an elongated body 131 having a rectangular cross-section, rectangular top and bottom surfaces and four rectangular sides 131a, 131b, 131c, 131d. The distal end portion 132 comprises an inverted truncated rectangular pyramid extending from the bottom surface of the elongated body 131 and the proximal end portion 134 comprises an inverted truncated rectangular pyramid extending from the top surface of the elongated body 131.

Referring to FIG. 4b, a new facet fixation and fusion device comprises a polygonal-shaped dowel 130 made of allograft bone material. The polygonal-shaped dowel 140 includes an elongated polygonal body 141, which in this case has a hexagonal cross-section, hexagonal top and bottom surfaces and six rectangular sides 141a, 141b, 141c, 141d, 141e, 141f. The distal end portion 142 comprises an inverted truncated polygonal pyramid extending from the bottom surface of the elongated body 141 and the proximal end portion 144 comprises an inverted truncated polygonal pyramid extending from the top surface of the elongated body 141. The edges 143 between two adjacent side surfaces (i.e., 141a, 141b or 131a, 131b) form a rigid and sharp edge that bites into the side walls of opening 192 in the facet joint.

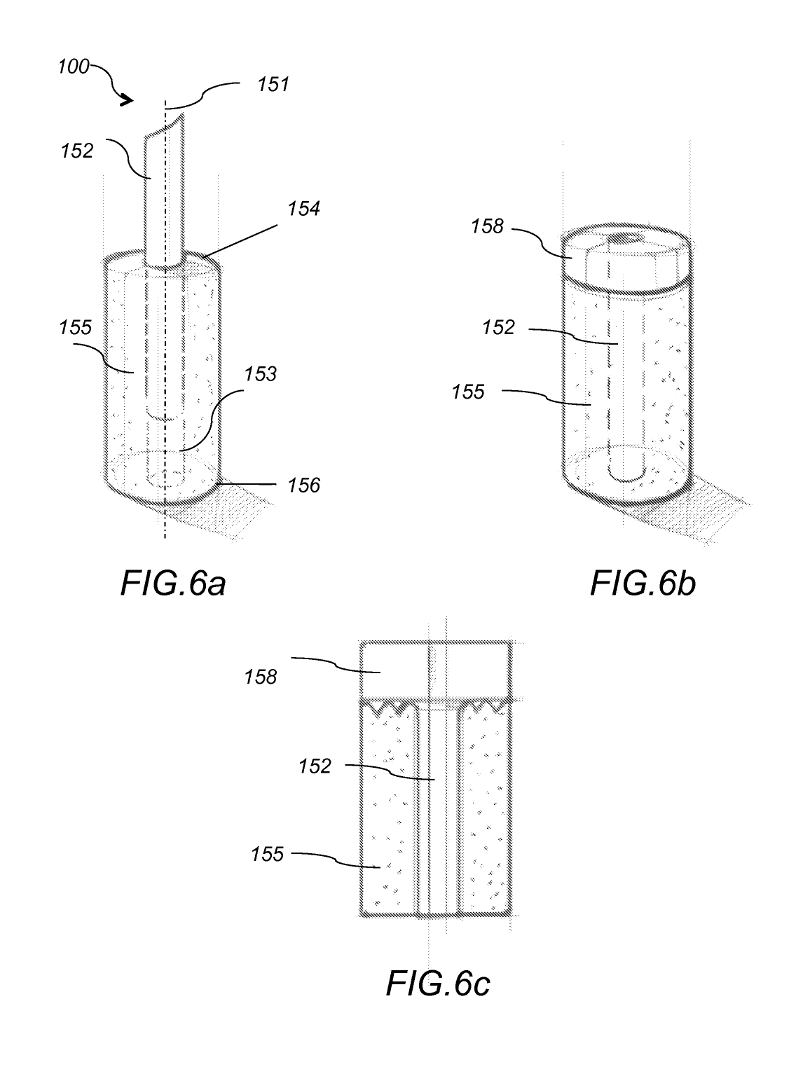

Referring to FIG. 6a, a new facet fixation and fusion device 150 comprises a combination of a cylindrical metallic rod 152 surrounded by an outer cylindrical body 155 made of allograft bone material. The outer cylindrical body 155 includes a through opening 153 extending along axis 151 from the proximal end 154 to the distal end 156. In the embodiment of FIG. 6b, the device 150 also includes a metal cap 158 that connects to the metal cylinder 152 inside the through opening 153, as shown in the cross-sectional view in FIG. 6C.

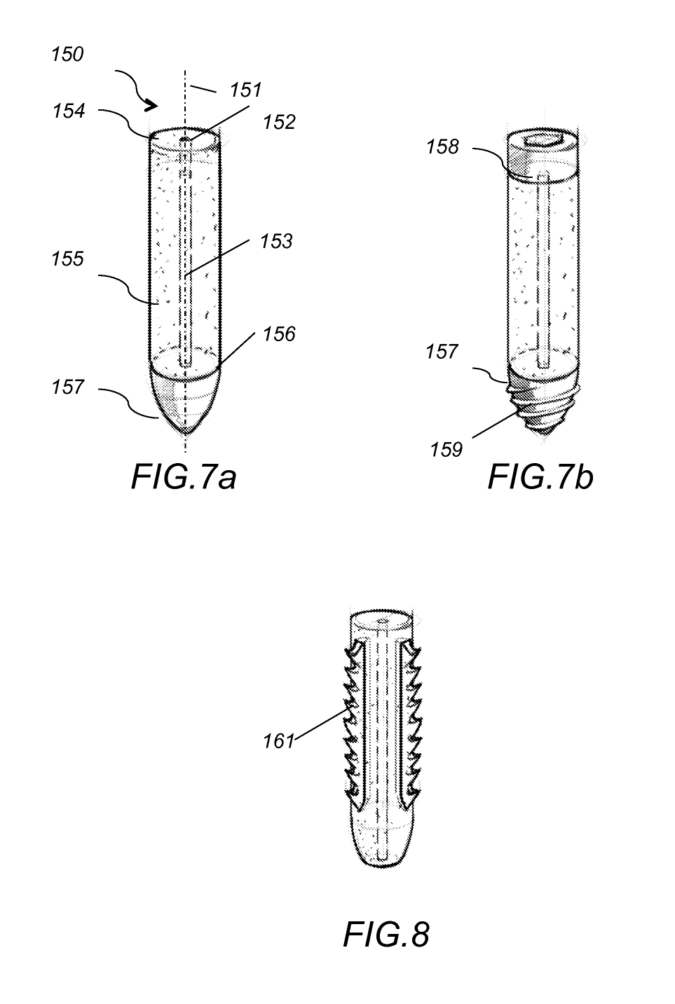

Referring to FIG. 7a, a new facet fixation and fusion device 150 comprises a cylindrical metallic rod 152 surrounded by an outer cylindrical body 155 made of allograft bone material. The device 150 also includes a “bulleted” conical shaped metallic cap 157 at the distal end of the cylindrical body 155. The outer cylindrical body 155 includes a through opening 153 extending along axis 151 from the proximal end 154 to the distal end 156.

In the embodiment of FIG. 7b, the device 150 also includes a metal cap 158 which connects to the metal cylinder 152 inside the through opening 153. In this embodiment the conical metallic cap 157 also includes screw threads 159 surrounding its outer surface. In the embodiment of FIG. 8, the cylindrical body 155 includes longitudinally extending metallic spikes or teeth 161 protruding from the sides of the cylindrical body 155.

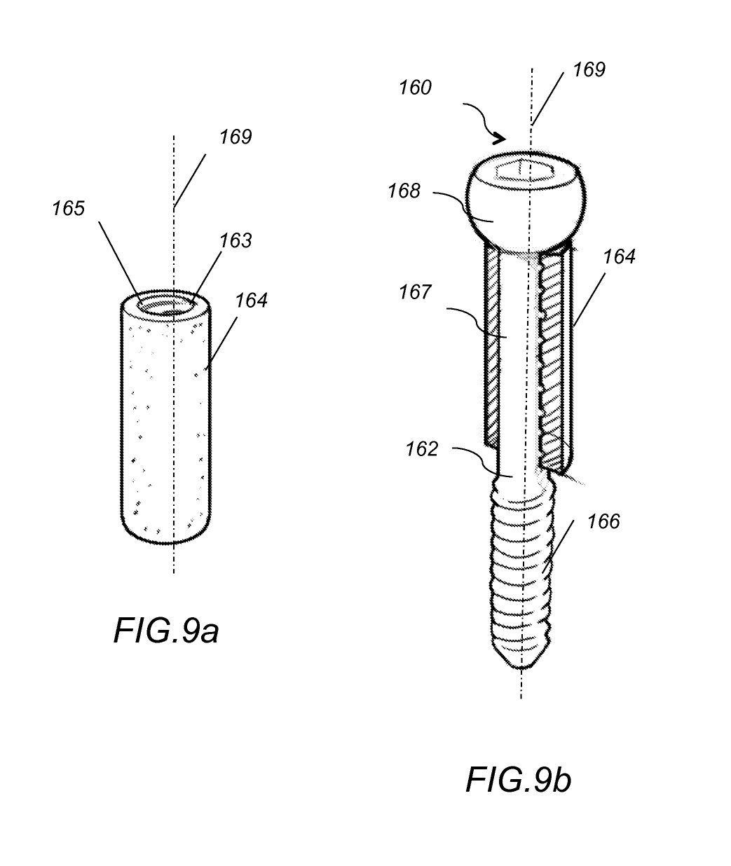

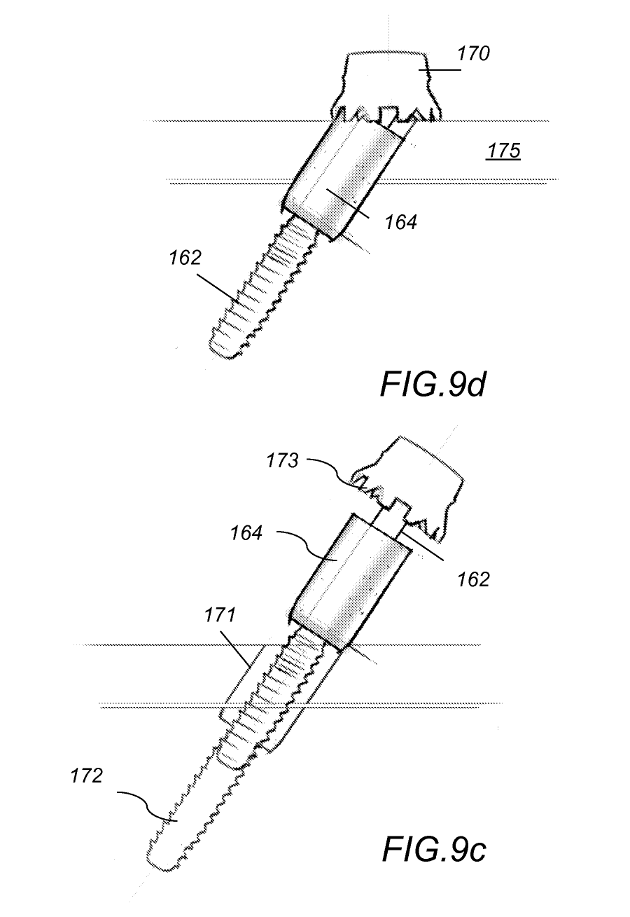

Referring to FIG. 9b, a new facet fixation and fusion device 160 comprises an elongated metallic screw 162 having a threaded distal portion 166, a head 168 and a lag portion 167 extending between the head 168 and the distal portion 166. The lag portion 167 is surrounded by an outer cylindrical body 164 made of allograft bone material. Cylindrical body 164 includes a through opening 165 extending along its longitudinal axis 169. Opening 165 includes inner threads 163. Fixation device 160 is placed in an opening 172 extending through the facet joints 175 and the opening 172 includes a countersink hole 171 at the top for housing the cylindrical body 164 and head 168 of the screw 162. In this embodiment screw 162 also includes a polyaxial washer 170 surrounding the head 168. Polyaxial washer 170 includes spikes 173 extending from its bottom surface. Spikes 173 are configured to engage the surrounding bone surface, as shown in FIG. 9d.

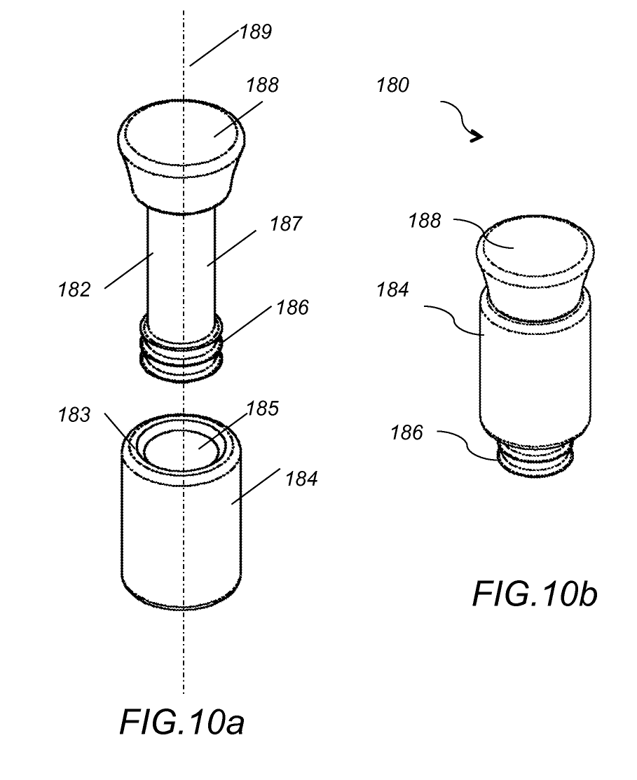

Referring to FIG. 10a, a new facet fixation and fusion device 180 comprises an elongated allograft plug 182 having a threaded distal portion 186, a head 188 and a lag portion 187 extending between the head 188 and the distal portion 186. The lag portion 187 is surrounded by an outer cylindrical body 184 also made of allograft bone material. Cylindrical body 184 includes a through opening 185 extending along its longitudinal axis 189. Opening 185 includes inner threads 183. Fixation device 180 is placed in an opening extending through the facet joints and the opening includes a countersink hole at the top for housing the cylindrical body 184 and head 188 of the plug 182, as was described above.

A first method of inserting the facet fixation and fusion device of FIG. 1a includes driving an opening 192 with an awl 194 through the inferior facet of vertebra 30b, facet joint 46b, and superior facet of vertebra 30a, with a trajectory towards the pedicle of the inferior vertebra 30a, as shown in FIG. 11a. Next, inserting a cannula 196 over the awl 194, removing the awl 194 and inserting a drill bit 198 through the cannula 196, as shown in FIG. 11b and FIG. 11C. Next, drilling a hole 192 with the drill bit 198 and then inserting the fixation device 100 into the hole 192 through the cannula 196 with the impacter 199. Finally removing the impacter 199 and cannula 196 leaving behind the inserted fixation device 100, as shown in FIG. 11e. The process is repeated for the other facet joint 46a. This method is applied for inserting any of the above described fixation devices 100, 110, 120, 130, 140, 150, 160, 180.

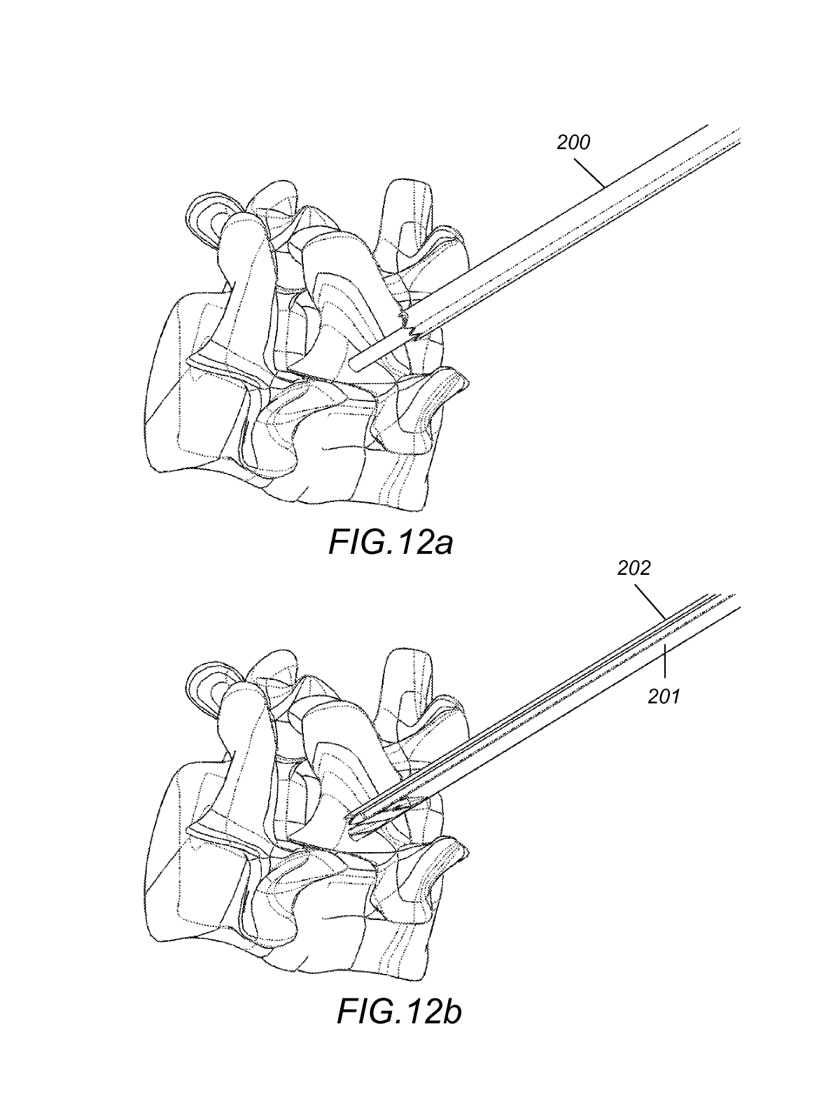

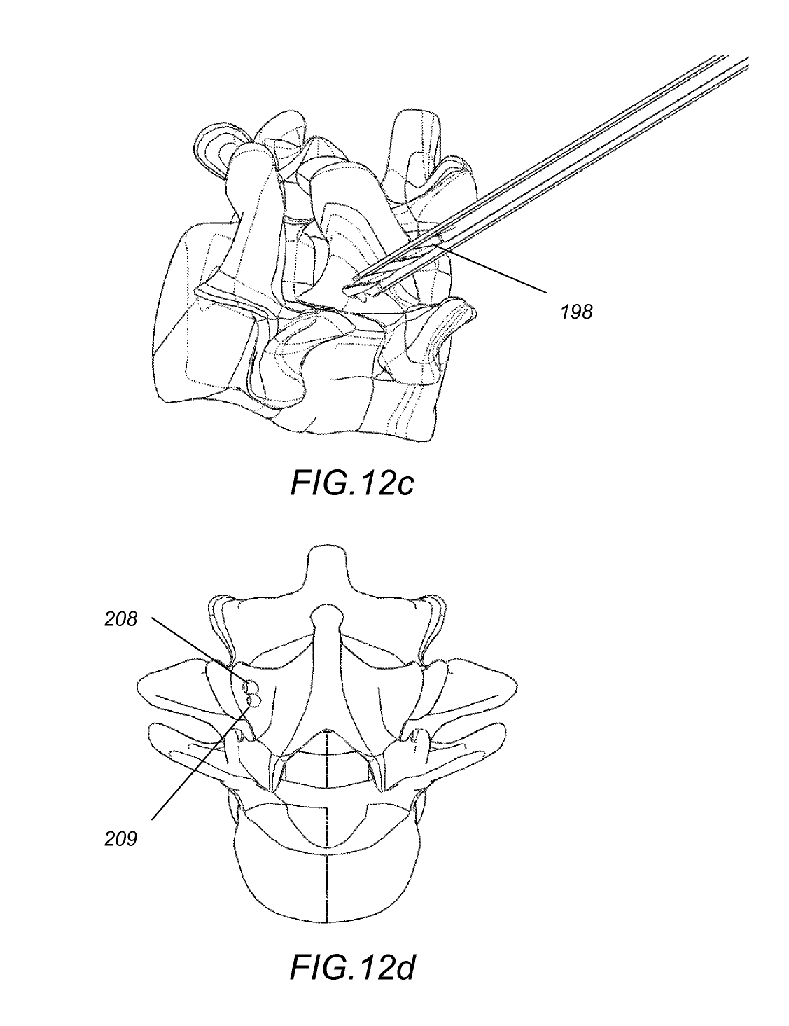

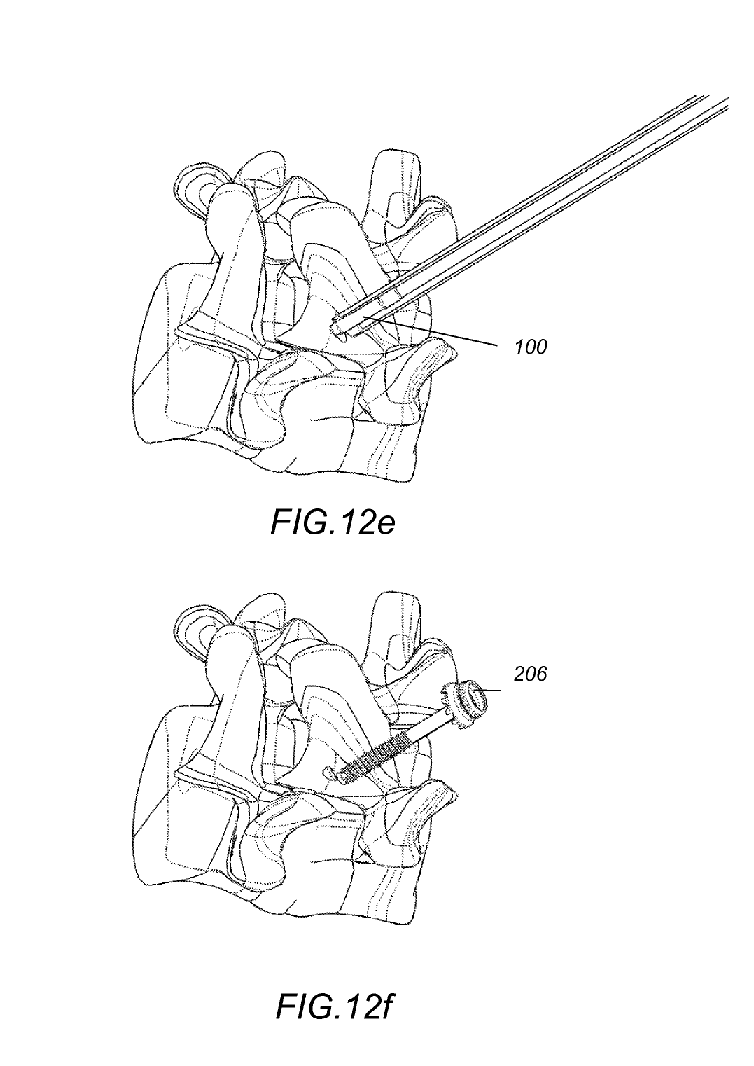

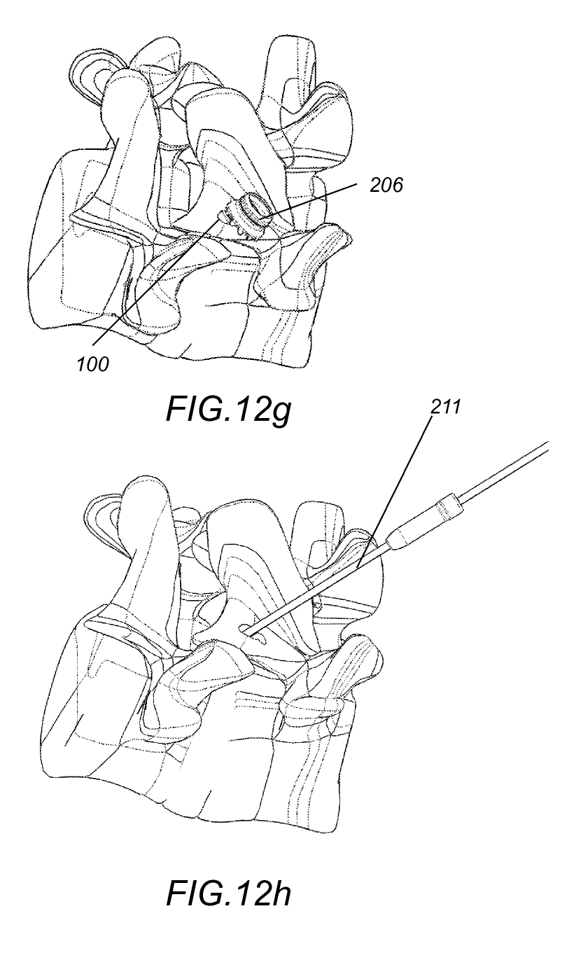

Referring to FIG. 12a-FIG. 12h, in a second method two adjacent openings 208, 209 are made instead of one. Openings 208, 209 may be intersecting each other or non-intersecting. The allograft fixation and fusion device 100 is inserted in opening 208 and a separate metallic facet screw 206 is inserted in the other opening 209, as shown in FIG. 12e and FIG. 12f, respectively. A double-barreled cannula 200 is used to reach the two openings. In the case of a cannulated allograft device 100 (shown in FIG. 1b), a guide wire 211 is used for inserting the allograft device in the opening 208, shown in FIG. 12h.

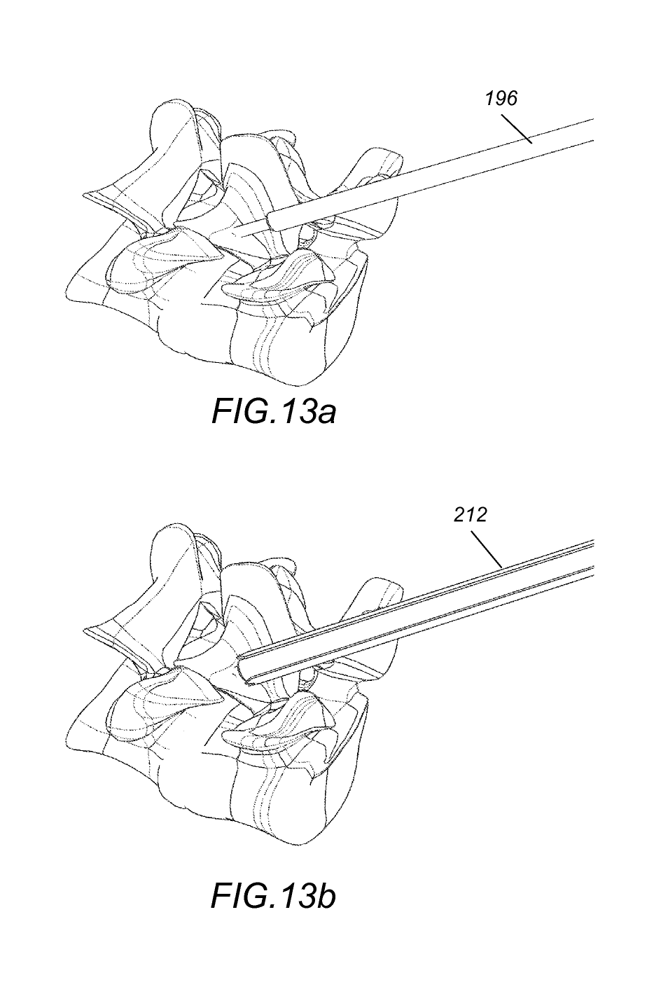

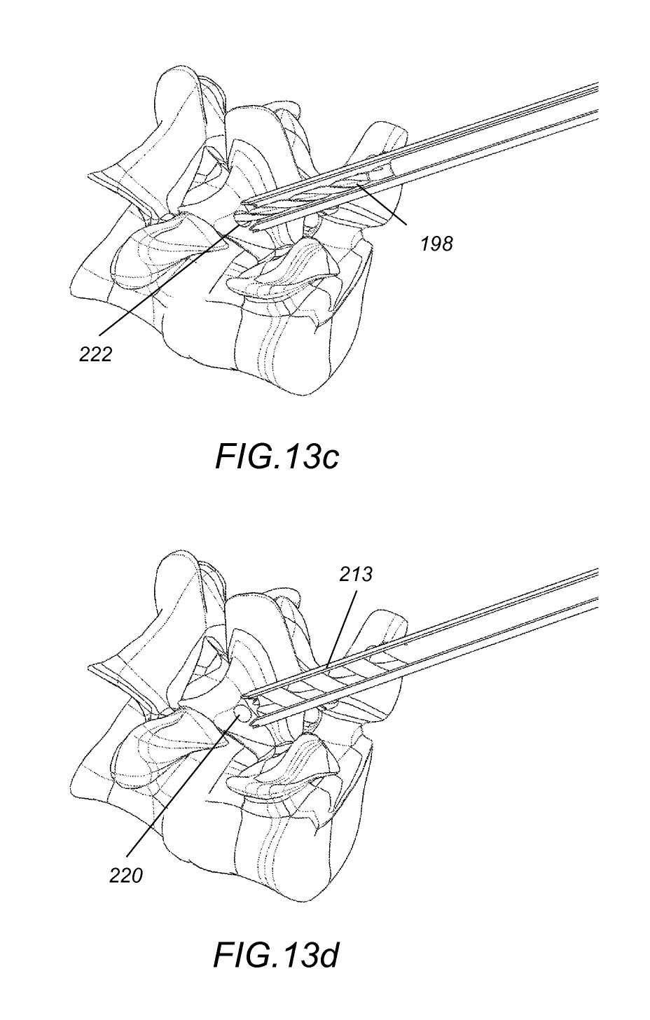

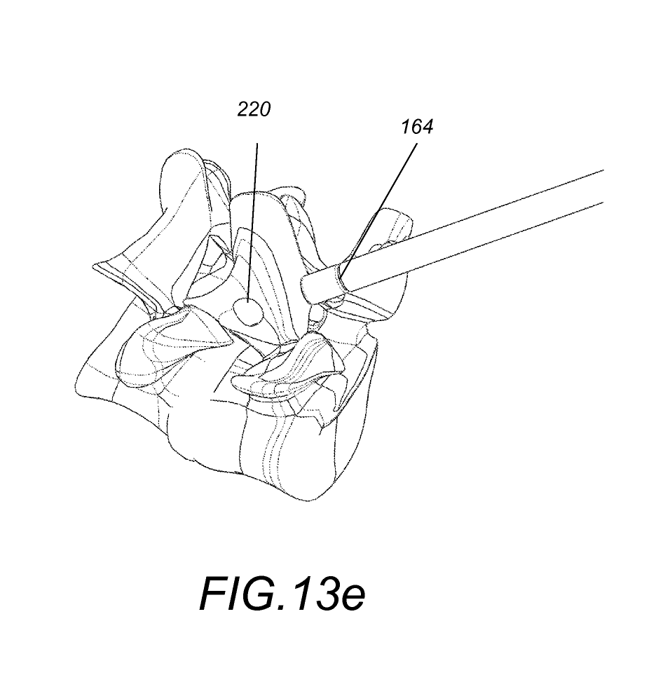

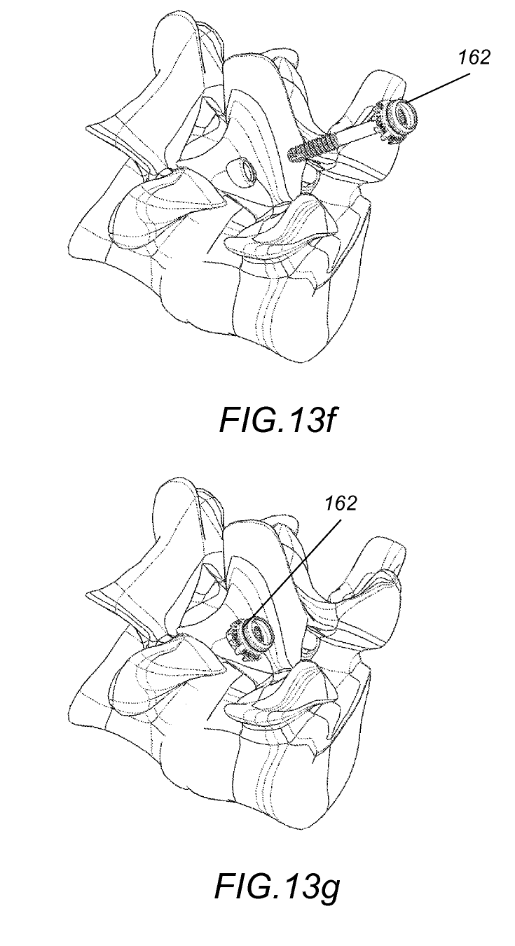

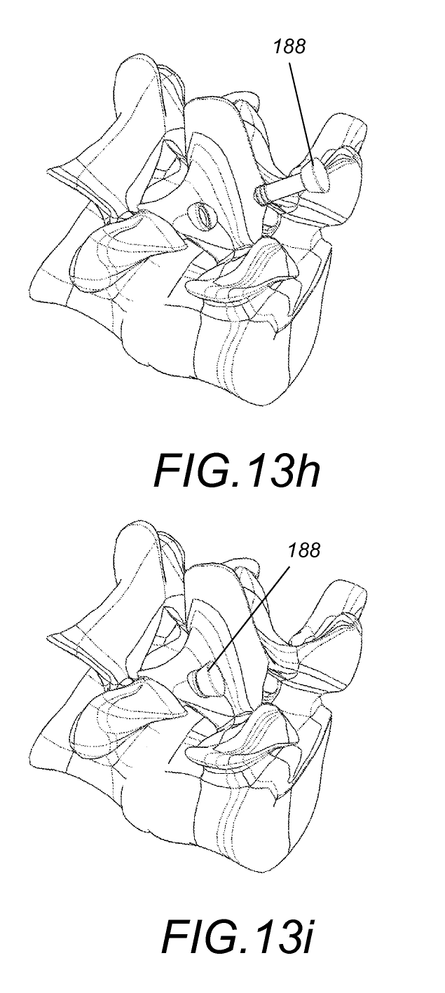

A third method is used for inserting the two component fixation and fusion device 160 or 180. Referring to FIG. 13a, first an opening is driven and a cannula 196 is inserted. Next, the opening is dilated with dilator 212, shown in FIG. 13b, and then a drill bit 198 is inserted through the cannula 196 for drilling a hole 222, shown in FIG. 13c. Next, a countersink hole 220 surrounding the top of the opening 222 is drilled, as shown in FIG. 13d and then the cylindrical allograft sleeve 164 is inserted in the countersink hole 220, as shown in FIG. 13e. Finally the metallic facet screw 162 or the allograft plug 188 is inserted in the cylindrical allograft sleeve 164, as shown in FIG. 13f or FIG. 13h, respectively.

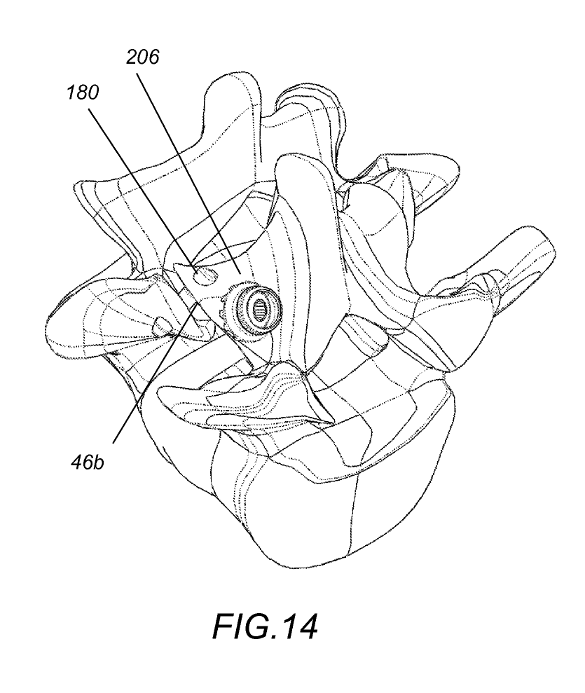

In another embodiment, two separate openings are drilled with different trajectories through the facet joint 46b, as shown in FIG. 14. In this embodiment, one opening is used for the allograft device 180 and the other opening is used for the metallic facet screw 206. The opening for the allograft device 180 has a more lateral trajectory extending through the facet joint 46b, as shown in FIG. 14.

Other embodiments include one or more of the following. The allograft bone material is substituted with other biocompatible materials including synthetic bone growth promoting material, bone-polymer composite material, autograft bone material, xenograft bone material, polymers, resorbable material, or non-resorbable material, or combinations thereof. The metallic components may be made of titanium, cobalt, stainless steel, chrome, or alloys thereof or shape-memory alloy, or ceramic-metallic composite materials, among others.

Several embodiments of the present invention have been described. Nevertheless, it will be understood that various modifications may be made without departing from the spirit and scope of the invention. Accordingly, other embodiments are within the scope of the following claims.

What is claimed is:

1. A spinal fixation and fusion assembly comprising:
a bone anchoring member comprising an elongated body, wherein said elongated body is comprised entirely of bone type material;
wherein said elongated body comprises a main shaft, a conical shaped distal end, a flared out proximal end, and a through-opening extending along an axis through the main shaft from the proximal end to the distal end;
a cylindrical rod, shaped and dimensioned to be received within said through-opening and being comprised entirely of metal; and
a cap being comprised entirely of metal and being attached to a proximal end of the cylindrical rod.

2. The spinal fixation and fusion assembly of claim 1 wherein said elongated body comprises one of cylindrical cross section, rectangular cross section, or polygonal cross section.

3. The spinal fixation and fusion assembly of claim 1 wherein said elongated body comprises one of screw threads, spikes, teeth, barbs, bumps, indentations, straight protrusions, helical protrusions, or combinations thereof.

4. The spinal fixation and fusion assembly of claim 1 wherein said main shaft comprises a parallelepiped shape, said distal end comprises an inverted truncated rectangular pyramid extending from the bottom surface of the main shaft, and said proximal end comprises an inverted truncated rectangular pyramid extending from the top surface of the main shaft.

5. The spinal fixation and fusion assembly of claim 1 wherein said main shaft comprises a polygonal shape, said distal end comprises an inverted truncated polygonal pyramid extending from the bottom surface of the main shaft, and said proximal end comprises an inverted truncated polygonal pyramid extending from the top surface of the main shaft.

6. The spinal fixation and fusion assembly of claim 1 further comprising a conical cap being comprised entirely of metal and being attached to a distal end of the cylindrical rod and wherein said conical cap comprises screw threads.

7. A spinal fixation and fusion assembly comprising:
a bone anchoring member comprising an elongated body, wherein said elongated body is comprised entirely of metallic material;
wherein said elongated body comprises a threaded portion at the distal end, a head at the proximal end, and a lag portion extending between the threaded portion and the head;
a cylindrical member surrounding said lag portion of the bone anchoring member and wherein said cylindrical member is comprised entirely of bone type material and comprises a through-opening extending from its proximal end to the distal end and wherein said through-opening comprises inner threads and is dimensioned to receive said lag portion of the bone anchoring member.

8. The spinal fixation and fusion assembly of claim 7 further comprising a polyaxial washer surrounding the head of the bone anchoring member, and wherein the polyaxial washer comprises spikes extending from its bottom surface.

9. A spinal fixation and fusion assembly comprising:
a bone anchoring member comprising an elongated body, wherein said elongated body is comprised entirely of bone type material;
wherein said elongated body comprises a threaded portion at the distal end, a head at the proximal end, and a lag portion extending between the threaded portion and the head;
a cylindrical member surrounding said lag portion of the bone anchoring member and wherein said cylindrical member is comprised entirely of bone type material and comprises a through-opening extending from its proximal end to the distal end and wherein said through-opening comprises inner threads, and is dimensioned to receive said lag portion of the bone anchoring member.

10. A spinal fixation and fusion method comprising:
forming an opening extending through first and second adjacent vertebral bodies;
inserting a spinal fixation and fusion assembly into said formed opening; and
wherein said spinal fixation and fusion assembly comprises a bone anchoring member, a cylindrical rod and a cap and wherein said bone anchoring member comprises an elongated body comprised entirely of bone type material and wherein said elongated body comprises a main shaft, a conical shaped distal end, a flared out proximal end, and a through-opening extending along an axis from the proximal end to the distal end, and wherein said cylindrical rod is shaped and dimensioned to be received within said through-opening and is comprised entirely of metal and wherein said cap is comprised entirely of metal and is attached to a proximal end of the cylindrical rod.

11. The spinal fixation and fusion method of claim 10 wherein said elongated body comprises one of cylindrical cross section, rectangular cross section, or polygonal cross section.

12. The spinal fixation and fusion method of claim 10 wherein said elongated body comprises one of screw threads, spikes, teeth, barbs, bumps, indentations, straight protrusions, helical protrusions, or combinations thereof.

13. The spinal fixation and fusion method of claim 10 wherein said main shaft comprises a parallelepiped shape, said distal end comprises an inverted truncated rectangular pyramid extending from the bottom surface of the main shaft, and said proximal end comprises an inverted truncated rectangular pyramid extending from the top surface of the main shaft.

14. The spinal fixation and fusion method of claim 10 wherein said main shaft comprises a polygonal shape, said distal end comprises an inverted truncated polygonal pyramid extending from the bottom surface of the main shaft, and said proximal end comprises an inverted truncated polygonal pyramid extending from the top surface of the main shaft.

15. The spinal fixation and fusion method of claim 10 wherein said spinal fixation assembly further comprises a conical cap being comprised entirely of metal and being attached to a distal end of the cylindrical rod and wherein said conical cap comprises screw threads.

16. A spinal fixation and fusion method comprising:
forming an opening extending through first and second adjacent vertebral bodies;
inserting a spinal fixation and fusion assembly into said formed opening; and
wherein said spinal fixation and fusion assembly comprises a bone anchoring member and a cylindrical member, wherein said bone anchoring member comprises an elongated body comprised entirely of metallic material and wherein said elongated body comprises a threaded portion at the distal end, a head at the proximal end, and a lag portion extending between the threaded portion and the head, and wherein said cylindrical member surrounds said lag portion of the bone anchoring member and wherein said cylindrical member is comprised entirely of bone type material and comprises a through-opening extending along an axis from its proximal end to the distal end and wherein said through-opening comprises inner threads and is dimensioned to receive said lag portion of the bone anchoring member.

17. The spinal fixation and fusion method of claim 16 wherein said spinal fixation and fusion assembly further comprises a polyaxial washer surrounding the head of the bone anchoring member, and wherein the polyaxial washer comprises spikes extending from its bottom surface.

18. A spinal fixation and fusion method comprising:
forming an opening extending through first and second adjacent vertebral bodies;
inserting a spinal fixation and fusion assembly into said formed opening; and
wherein said spinal fixation and fusion assembly comprises a bone anchoring member and a cylindrical member, wherein said bone anchoring member comprises an elongated body comprised entirely of bone type material and wherein said elongated body comprises a threaded portion at the distal end, a head at the proximal end, and a lag portion extending between the threaded portion and the head, and wherein said cylindrical member surrounds said lag portion of the bone anchoring member and wherein said cylindrical member is comprised entirely of bone type material and comprises a through opening extending along an axis from its proximal end to the distal end and wherein said through opening comprises inner threads and is dimensioned to receive said lag portion of the bone anchoring member.

19. A facet fixation and fusion method comprising:
forming a first opening extending through a facet joint of first and second adjacent vertebral bodies;
forming a second opening extending through said first and second adjacent vertebral bodies, wherein said second opening is adjacent to said first opening;
inserting a first spinal fixation assembly into said first opening;
inserting a second spinal fixation and fusion assembly into said second opening;
wherein said first fixation assembly comprises a bone anchoring member and a polyaxial washer and wherein said bone anchoring member comprises an elongated body having a threaded portion at the distal end, a head at the proximal end, and a lag portion extending between the threaded portion and the head and wherein said polyaxial washer surrounds said head, and wherein said first fixation assembly is comprised entirely of metallic material; and
wherein said second fixation and fusion assembly comprises a cylindrical elongated body comprised entirely of bone type material.

20. The facet fixation and fusion method of claim 19, wherein said first and second openings intersect each other.

21. The facet fixation and fusion method of claim 19, wherein said first and second openings do not intersect each other.

22. The facet fixation and fusion method of claim 19, wherein said first and second openings comprise parallel trajectories.

23. The facet fixation and fusion method of claim 19, wherein said first and second openings comprise non-parallel trajectories.

24. The facet fixation and fusion method of claim 19, wherein said first and second openings are formed through a double-barreled cannula.

25. The spinal fixation and fusion assembly of claim 1, wherein said bone type material comprises one of allograft bone material, biocompatible materials, synthetic bone growth promoting material, bone-polymer composite material, autograft bone material, xenograft bone material, polymers, resorbable material, non-resorbable material, or combinations thereof.

26. The spinal fixation and fusion of claim 1, wherein said metal comprises one of titanium, cobalt, stainless steel, chrome, alloys thereof, shape-memory alloy, ceramic-metallic composite materials, or combinations thereof.
